(19) United States
(12) Patent Application Publication
(10) Pub. No.: US 2022/0392593 A1
(43) Pub. Date: Dec. 8, 2022

(54) Medical Surgery Recording, Processing and Reporting System

(71) Applicant: Mirza Faizan, Irving, TX (US)

(72) Inventors: Mirza Faizan, Irving, TX (US); Mirza Rizwan, Patna (IN); Nabeel Abdul Rahman, Raleigh, NC (US); Ayesha Badar, Raleigh, NC (US); Nabeel Balighuddin, Morrisville, NC (US); Aiman Fatima Jamadar, Cary, NC (US); Nava Alam Mazumder, Cary, NC (US); Adnan Murtaza Shaikh, Raleigh, NC (US); Husaam Murtaza Shaikh, Raleigh, NC (US); Humairaa Ayesha Zafiruddin, Raleigh, NC (US)

(21) Appl. No.: 17/339,508

(22) Filed: Jun. 4, 2021

(51) Int. Cl. G16H 15/00; G06K 9/00; G06K 9/62; H04N 7/18; G16H 30/40

(52) U.S. Cl. CPC G16H 15/00; G06K 9/00335; G06K 9/00718; G06K 9/6215; H04N 7/183; G16H 30/40; G06K 2209/057

(57) ABSTRACT

The present invention directed to a medical reporting system. The system comprising a fixed visual capturing unit integrated at a first location to capture one or more first set of visuals and a movable visual capturing unit integrated at a second location to capture one or more second set of visuals. The system further comprising a separator to separate the stored one or more first set of visuals and the stored one or more second set of visuals in plurality of frames of pre-defined length. The system further comprising a first database unit and a second database unit. The system further comprising a comparator configured to compare each of the separated plurality of frames. The system further comprising a processor to process a positive output of the comparator and a transceiver to communicate the positive output to a transcription unit for medical reporting.

### TECHNICAL FIELD

The invention relates to a surgical image processing. More specifically, the invention relates to reporting system by the processed surgical image. Furthermore specifically, the invention relates to a readable report generating system by processing surgical images.

### BACKGROUND OF THE INVENTION

Today, with a large population the number of patients in the medical facilities across the globe has been significantly increased. It has been noted that the number of doctors present in the world are much less than the total required doctors. Hence, the doctors or the medical practitioners, are suffering from heavy mental pressure of treating a large number of patients and hence they are unable to take proper rest. This further leads to deterioration of their health issues and impacting personal life too.

It has been further noted that much of a doctor's time goes towards writing surgical reports after the operation. Writing these operative reports is a long, time-consuming task that the doctor must complete for each individual patient and is practically very difficult to write detailed operative reports. Presently, the devices available to convert the medical reports lack in accuracy and furthermore are not able to recognize the entire video.

Furthermore, the accuracy of the medical record is of the utmost importance. The medical record describes the patient's medical history, which may be of critical importance in providing future healthcare to the patient. Further, the medical record may also be used as a legal document, as a research tool and to provide information to insurance companies or third party reimbursors. Thus while writing the report of a patient, the doctor or the medical practitioner may mistakenly forget to mention an important point, which may lead to future health issues.

Hence there is an utmost need to build an artificial intelligence based system to capture frames, analyze, and transcribe the operation. And henceforth building a system to make writing operative reports less time-consuming and to mitigate errors.

### SUMMARY OF THE INVENTION

An embodiment is directed to a medical reporting system. The system comprising a fixed visual capturing unit integrated at a first location configured to capture one or more first set of visuals. The system further comprising movable visual capturing unit integrated at a second location configured to capture one or more second set of visuals. The system further comprising a memory unit configured to store the captured one or more first set of visuals and the captured one or more second set of visuals. The system further comprising a separator configured to separate the stored one or more first set of visuals and the stored one or more second set of visuals in plurality of frames of pre-defined length. The system further comprising a first database unit configured to store plurality of images related to surgical devices. The system further comprising a second database unit configured to store plurality of actions. The system further comprising a comparator configured to compare each of the separated plurality of frames, the stored plurality of images related to surgical devices and the stored plurality of actions; and the system further comprising a processor configured to: process a positive output of the comparator; wherein the positive output is a similar match found between the surgical devices or the action in a frame from each of the separated plurality of frames and the stored plurality of images related to surgical devices or the stored plurality of actions. The system further comprising a transceiver configured to communicate the positive output to a transcription unit, wherein the transcription unit is configured to transcript the communicated matched positive output for medical reporting.

In addition to one or more of the features described above or below, or as an alternative, further comprising a display module.

In addition to one or more of the features described above or below, or as an alternative, wherein the first location and the second location are predetermined locations.

In addition to one or more of the features described above or below, or as an alternative, wherein the fixed visual capturing unit is a rectangular prism-shaped unit.

In addition to one or more of the features described above or below, or as an alternative, wherein the movable visual capturing unit is an oval shaped unit.

In addition to one or more of the features described above or below, or as an alternative, wherein the processor is further configured to determine non positive outputs.

In addition to one or more of the features described above or below, or as an alternative, further comprising a content writing module.

An embodiment is directed to medical reporting method. The method comprising capturing one or more first set of visuals. The method further comprising capturing one or more second set of visuals. The method further comprising storing the captured one or more first set of visuals and the captured one or more second set of visuals. The method further comprising separating the stored one or more first set of visuals and the stored one or more second set of visuals in plurality of frames of pre-defined length. The method further comprising storing plurality of images related to surgical devices. The method further comprising storing plurality of actions. The method further comprising comparing each of the separated plurality of frames, the stored plurality of images related to surgical devices and the stored plurality of actions. The method further comprising processing a positive output of the comparison. In an embodiment, the positive output is a similar match found between the surgical devices or the action in a frame from each of the separated plurality of frames and the stored plurality of images related to surgical devices or the stored plurality of actions. The method further comprising communicating the positive output to a transcription unit; and the method further comprising transcripting the communicated matched positive output for medical reporting.

In addition to one or more of the features described above or below, or as an alternative, wherein the method further comprising displaying one or more first set of visuals in real time.

In addition to one or more of the features described above or below, or as an alternative, wherein the method further comprising displaying one or more second set of visuals in real time.

In addition to one or more of the features described above or below, or as an alternative, further comprising sorting the compared frames based on the positive outputs.

In addition to one or more of the features described above or below, or as an alternative, further comprises generating a medical report for the medical reporting.

In addition to one or more of the features described above or below, or as an alternative, further comprising generating medical database of a user based on communicated medical reporting.

In addition to one or more of the features described above or below, or as an alternative, further comprising comparing generated medical database of the user with a historical record of the user to record one or more effects of treatments.

In addition to one or more of the features described above or below, or as an alternative, further comprises writing the medical report in one or more user friendly language for medical reporting.

### OBJECTIVES OF THE INVENTION

The objective of the disclosed invention is to provide a detailed medical report of the medical actions performed on a user.

Yet another objective of the invention is to generate the detailed medical report with the use of artificial intelligence and further to store the medical history of a patient in the event of an emergency in the device.

Yet another objective of the present invention is to remove the complex process of preparing detailed reporting of the user, by the disclosed method and thereby reducing the time required to prepare the report.

Yet another objective of the invention is to stream the medical actions performed on the user to one or more other users by using wireless means.

Yet another objective of the present invention is to automatically detect the newly added content in the report of the user by a medical practitioner.

### BRIEF DESCRIPTION OF THE DRAWINGS

The accompanying drawings illustrate various embodiments of systems, methodₛ, and other aspects of the disclosure. Any person having ordinary skill in the art will appreciate that the illustrated element boundaries (e.g., boxes, groups of boxes, or other shapes) in the figures represent one example of the boundaries. It may be that in some examples, one element may be designed as multiple elements or that multiple elements may be designed as one element. In some examples, an element shown as an internal component of one element may be implemented as an external component in another, and vice versa. Furthermore, elements may not be drawn to scale.

Various embodiments will hereinafter be described in accordance with the appended drawings, which are provided to illustrate, and not to limit the scope in any manner, wherein like designations denote similar elements, and in which:

FIG. 1 is a block diagram illustrating a system environment in which various embodiments may be implemented;

FIG. 2 is an exemplary block diagram of a system, in accordance with at least one embodiment.

FIG. 3 is a flowchart illustrating a method for surgical image processing and reporting system, in accordance with at least one embodiment; and

FIG. 4 is an exemplary scenario for surgical image processing and reporting system, in accordance with at least one embodiment.

### DETAILED DESCRIPTION OF DRAWINGS

The present disclosure is best understood with reference to the detailed figures and description set forth herein. Various embodiments are discussed below with reference to the figures. However, those skilled in the art will readily appreciate that the detailed descriptions given herein with respect to the figures are simply for explanatory purposes as the methods and systems may extend beyond the described embodiments. For example, the teachings presented and the needs of a particular application may yield multiple alternate and suitable approaches to implement the functionality of any detail described herein. Therefore, any approach may extend beyond the particular implementation choices in the following embodiments described and shown.

References to “one embodiment,” “an embodiment,” “at least one embodiment,” “one example,” “an example,” “for example,” and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment.

Definitions: The following terms shall have, for the purposes of this application, the respective meanings set forth below.

A “computing device” refers to a device that includes one or more processors/microcontrollers and/or any other electronic components, or a device or a system that performs one or more operations according to one or more programming instructions/codes. Examples of a computing device may include, but are not limited to, a desktop computer, a laptop, a personal digital assistant (PDA), a mobile device, a smartphone, a tablet computer (e.g., iPad®, and Samsung Galaxy Tab®), and the like.

A “patient or a user” is a human being who may require medical care or treatment by a medical expert, such as a doctor. In other words, a patient is a recipient of health care services provided by a health practitioner. In an embodiment, a patient refers to a patient who is currently under medical observation.

An “electronic medical record” refers to a documentation of health condition of a patient. In an embodiment, the medical record may include periodic measures of physiological parameters associated with the patient. Further, the medical record may include nursing notes documented over a specific time by a healthcare professional (such as a doctor, a nurse, a medical attender, a hospital staff, and/or the like). In an embodiment, the nursing notes may include recorded observations, administered drugs and therapies, test results, X-rays, nursing reports, investigative reports, and the like. In an embodiment, the medical record may be documented on a computing device, such as, but not limited to, a desktop computer, a laptop, a PDA, a mobile device, a smartphone, a tablet computer (e.g., iPad® and Samsung Galaxy Tab®), and the like. In an embodiment, the medical record may correspond to electronic or handwritten document(s).

A “nursing note of vitals” refers to a medical record that may describe a health condition of a patient and an administered or planned treatment. The nursing note may be documented by a nurse, physician, and other healthcare professionals for recording the health condition of the patient. The nursing note may comprise prescribed treatments, response to the prescribed treatments, medical diagnosis, and/or the like. The nursing note, corresponding to the patient, may be recorded on a daily or periodic basis. Hereinafter, “nursing note” and “vitals report” may be interchangeably used.

“Historical data” refers to one or more medical records or vitals of one or more users who were under medical observations in the past. In an embodiment, the one or more medical records may comprise a measure of one or more physiological parameters (e.g., blood pressure, heart rate, respiratory rate, body temperature, and the like) associated with the one or more patients. Further, the one or more medical records may comprise lab investigation data (e.g., a sodium level, a potassium level, a glucose level, and the like), diagnostics data, and other medical data associated with the one or more patients.

A “sensor” refers to a device that detects/measures events or changes in quantities and provides a corresponding output, generally as an electrical or optical signal. In healthcare domain, a first type of sensors may be operable to detect and measure various biological and physical variations corresponding to the patient. Such detected and measured signals may be recorded for further analytics. For example, biomedical sensors are used to monitor heart rate, respiration rate, pulse rate, blood pressure, and the like, of the first patient. Further, sensors may be operable to detect and measure various physical and/or chemical signals corresponding to a medical device associated with the patient. For example, pressure sensors, temperature sensors, and humidity sensors are used to monitor and regulate gas flow and gas conditions in anesthesia machines, respirators, and ventilators. The sensor may be an acceleration sensor or a vibration sensor, such as a VTT or TI standard chip base accelerometer. These examples are currently contemplated, but it should be understood that alternatives exist.

A “fixed visual capturing unit” refers to a video camera which may be placed above the operation table. In an embodiment of the present invention, the fixed visual capturing unit may be a rectangular prism-shaped unit.

A “movable visual capturing unit” refers to a video camera which may be placed on the upper portion of the medical practitioner. In an embodiment of the present invention, the fixed visual capturing unit may be an oval-shaped unit.

As used herein, the terms “images,” “records,” “medical reports” and “medical files” may be used interchangeably, and the foregoing terms comprise without limitation medical images, medical records, medical reports, medical files, body dimension data, studies, any type of laboratory reports and/or results, pathology images, reports, and results, hospital notes, consults, or any other type of data, report, or results that are stored in a hospital information system (HIS), and other medical data or other patient data. The foregoing terms can also include without limitation data, reports, and/or results generated in the ambulatory setting, for example, electronic medical record (EMR) to EMR communications, or information transferred, generated, created, and/or accessed in relay health type systems that manage patient communications, and other type of medical messaging and/or communication between any types of systems.

The terms “hospital” or “medical facility” as used herein are interchangeably, and both terms comprise without limitation hospitals, private doctors' offices, medical imaging facilities, clinics, emergency and/or urgent care centers, mobile care centers, medical kiosk stations, computer stations of medical professionals, both at homes and at offices, and other medical facilities. In certain embodiments, the term “medical facility” also comprises retail outlets (both online and physical retail stores), manufacturers, and the like. In certain embodiments, the term “medical facility” also comprises but is not limited to third party individuals, consultants, contractors, and/or outsourcing facilities.

As used herein the terms “medical practitioners”, “medical personnel” or “medical professional” are interchangeably used herein, and the foregoing terms comprise but are not limited to personnel that store and control access to patient medical image and/or record files, doctors, nurses, medical staff, physician aids, medical secretaries, physician assistants, or any other medical professional with access and/or authorization to create, locate, and/or match patient medical images and/or record files.

The terms “client server system,” “remote server system,” and “medical data transfer server” are broad interchangeable terms, and refer without limitation to computing systems and/or to systems that are involved in the process of processing and/or transferring medical files or the like, and/or controlling a workflow process for processing and/or transferring medical files. In certain embodiments, such computing systems are located at the medical facility and can communicate with the medical facility systems, such as PACS, RIS, HIS, and the like. Alternatively, such computing systems are located at a central facility and/or a hosting facility and/or a third party facility that may be located separate and apart from the medical facility. In certain embodiments, the computing system can act as a virtual remote server system that can communicate with the systems (for example, PACS, RIS, HIS, and the like) at and/or connected to the medical facility that is being served by the remote server system.

It is recognized that the term “remote” may include data, objects, devices, components, and/or modules not stored locally, that is not accessible via the local bus. Thus, remote data may include a device which is physically stored in the same room and connected to the computing system via a network. In other situations, a remote device may also be located in a separate geographic area, such as, for example, in a different location, country, and so forth.

The terms “message,” “medical system message,” and “medical system communication” are broad interchangeable terms, and refer without limitation to information and/or messages sent between and/or to hospital systems, for example, RIS, HIS, PACS, image servers, or other hospital systems. For example, a medical system message may include but are not limited to EMR to EMR communications, or information transferred, generated, created, and/or accessed in relay health type systems that manage patient communications, and other type of medical messaging and/or communication between any types of systems.

It is noted that various connections are set forth between elements in the following description and in the drawings (the contents of which are included in this disclosure by way of reference). It is noted that these connections in general and, unless specified otherwise, may be direct or indirect and that this specification is not intended to be limiting in this respect. In this respect, a coupling between entities may refer to either a direct or an indirect connection.

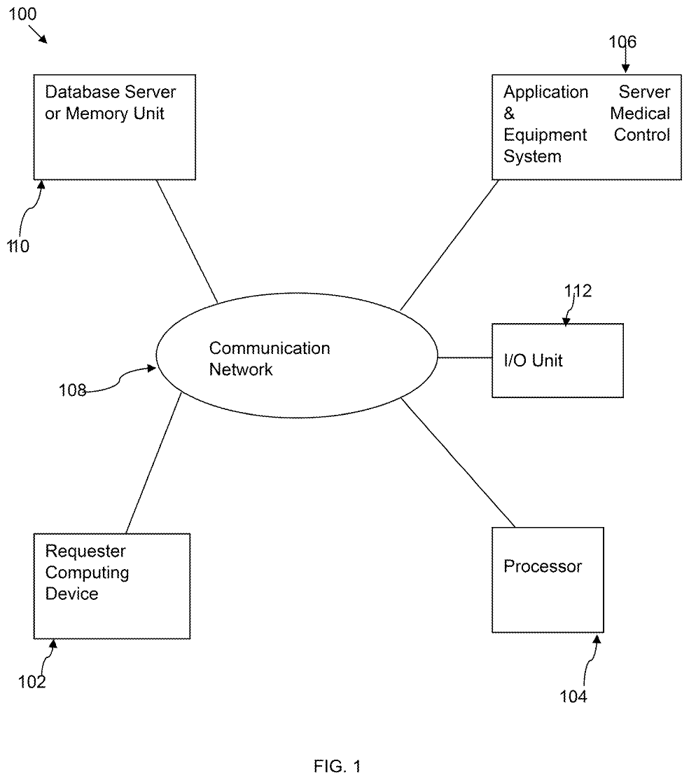

FIG. 1 is a block diagram illustrating a system environment in which various embodiments may be implemented. FIG. 1 shows a system environment 100 that includes a requestor-computing device 102, a processor 104, an application server and medical equipment control system 106, a communication network 108, and a database server (memory unit) 110. Various devices in the system environment 100 may be interconnected over the communication network 108. FIG. 1 shows, for simplicity, one requestor-computing device, such as the requestor-computing device 102, one processor, such as the processor 104, one application server, such as the application server 106, and one database server, such as the database server (memory unit) 110. However, it will be apparent to a person having ordinary skill in the art that the disclosed embodiments may also be implemented using multiple requestor-computing devices, multiple database servers, and multiple applications servers, without deviating from the scope of the disclosure.

The requestor-computing device 102 refers to a computing device that may comprise one or more processors in communication with one or more memories. The requestor-computing device 102 may be operable to execute one or more sets of instructions stored in the one or more memories. In an embodiment, the requestor-computing device 102 may be communicatively coupled with the communication network 108.

The requestor-computing device 102 may be used by a requestor, such as a medical practitioner to transmit/receive one or more positive output of the separated plurality of frames. In an embodiment, the similar match is found between the surgical devices or the action in a frame from each of the separated plurality of frames and the stored plurality of images related to surgical devices or the stored plurality of actions.

In an embodiment, the requestor may utilize the requestor-computing device 102 to provide one or more input parameters to perform one or more operations, such as, but not limited to, capturing a direct image for capturing one or more first set of visuals and/or one or more second set of visuals. In an embodiment, the requestor-computing device 102 may be coupled with the medical equipment control system 106. In an embodiment, the requestor-computing device 102 may comprise a display screen that may be configured to display one or more user interfaces to the requestor.

The requestor-computing device 102 may correspond to various types of computing devices, such as, but not limited to, a desktop computer, a laptop, a PDA, a mobile device, a smartphone, a tablet computer, an electronic console and the like.

Processor 104 may communicate with a user through control interface and display interface coupled to a display. The display may be, for example, a TFT LCD (Thin-Film-Transistor Liquid Crystal Display) or an OLED (Organic Light Emitting Diode) display, or other appropriate display technology. The display interface may comprise appropriate circuitry for driving the display to present graphical and other information to a user. The control interface may receive commands from a user and convert them for submission to the processor 104. In addition, an external interface may be provided in communication with processor 104, so as to enable near area communication of plurality of devices in the entire system. External interface may provide, for example, for wired communication in some implementations, or for wireless communication in other implementations, and multiple interfaces may also be used.

In an embodiment of the present invention, the processor 104 may be configured to process a positive output of the comparator. The positive output is a similar match found between the surgical devices or the action in a frame from each of the separated plurality of frames and the stored plurality of images related to surgical devices or the stored plurality of actions.

In an embodiment, the written notes of the medical practitioner may be processed by the processor 104. These written notes may correspond to the detailed notes of the action performed by the medical practitioner on the patient which may have not matched with nay of the sored one or more frames in the memory unit 110.

The application server 106 may refer to a computing device or a software framework that may provide a generalized approach to create the application server on a computer. In an embodiment, the function of the application server 106 may be dedicated to the efficient execution of procedures, such as, but not limited to, programs, routines, or scripts stored in one or more memories for supporting applied applications.

In an embodiment, the application server 106 may be accessed by the requestor-computing device 102, over the communication network 108, to receive the positive output or one or more other frames. Alternatively, the application server 106 may extract the one or more stored frames and/or videos of a patient (that are pre-stored) from the database server 110. The application server 106 may be realized using various technologies such as, but not limited to, Java application server, .NET Framework, PHP, Base4 application server, and Appserver.

The communication network 108 corresponds to a medium through which requests, content (such as one or more frames, videos, records), and messages may flow between the requestor-computing device 102, the database server 110, and the application server 106. Examples of the communication network 108 may include, but are not limited to, a Wireless Fidelity (Wi-Fi) network, a Wide Area Network (WAN), a Local Area Network (LAN), or a Metropolitan Area Network (MAN). Various devices, such as the requestor-computing device 102, the database server 110, and the application server 106, may connect to the communication network 108, in accordance with various wired and wireless communication protocols, such as Transmission Control Protocol/Internet Protocol (TCP/IP), User Datagram Protocol (UDP), and 2G, 3G, or 4G communication protocols.

In an embodiment, the communication network 108 may be configured to send/receive medical system messages and/or to send and receive a real time confirmation of patient's guardian in case of an emergency.

The database server or memory unit 110 may refer to a computing device that may store a repository of historical records of one or more frames of one or more patients. In an embodiment, the database server 110 may store metadata pertaining to the historical medical records of the one or more patients. For querying the database server 110, one or more querying languages may be utilized such as, but not limited to, structured query language (SQL), relational database query language (QUEL), data mining extensions (DMX), and so forth. Further, the database server 104 may be realized through various technologies such as, but not limited to, Microsoft® SQL server, Oracle®, and MySQL®.

The I/O unit 112 may comprise suitable logic, circuitry, interfaces, and/or code that may be configured to receive an input or transmit an output to the requestor-computing device 102. The input/output unit 112 comprises various input and output devices that are configured to communicate with the processor 104. Examples of the input devices include, but are not limited to, a keyboard, a mouse, a joystick, a touch screen, a microphone, a camera, and/or a docking station. Examples of the output devices include, but are not limited to, a display screen and/or a speaker.

The system 100 is illustrative. In some embodiments, one or more of the entities may be optional. In some embodiments, additional entities not shown may be included. For example, in some embodiments the system 100 may be associated with one or more networks. In some embodiments, the entities may be arranged or organized in a manner different from what is shown in FIG. 1.

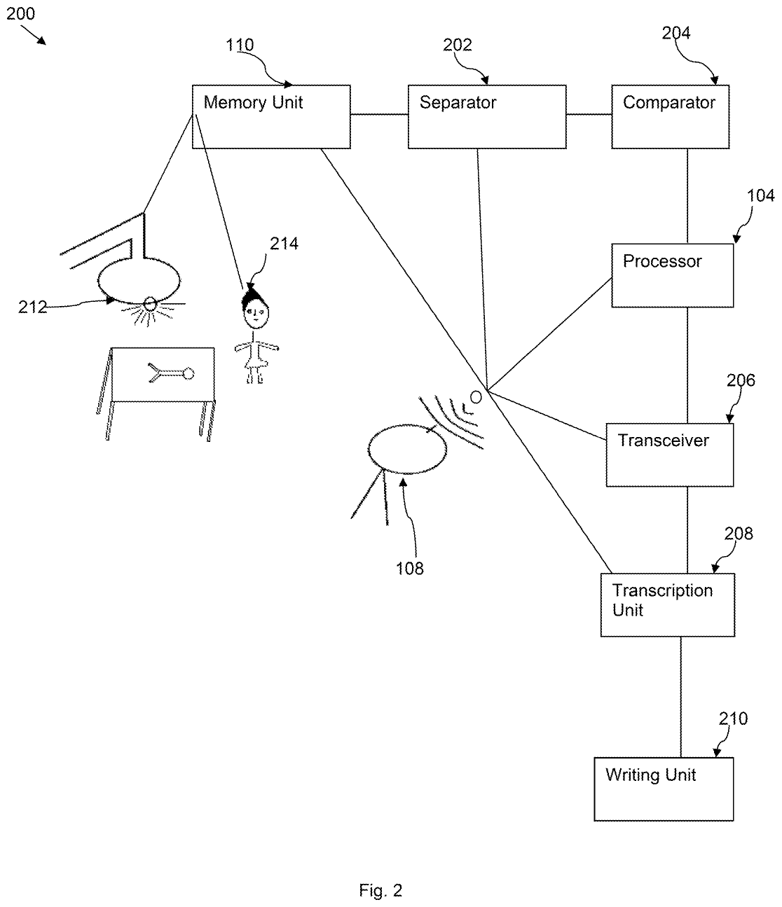

FIG. 2 is an exemplary block diagram of a system, in accordance with at least one embodiment. With reference to FIG. 2, there is shown a system 200 that may include the memory unit 110, a separator 202, a comparator 204, the processor 104, a transceiver 206, a transcription unit 208, the communication network 108, and a writing unit 210. FIG. 2 shows, for simplicity, one separator, such as the a separator 202, one comparator, such as the comparator 204, one transceiver, such as the transceiver 206, one transcription unit, such as the transcription unit 208, one writing unit, such as the writing unit 210, one fixed image capturing unit, such as the fixed visual capturing unit 212, and one movable visual capturing unit, such as the movable visual capturing unit 214. However, it will be apparent to a person having ordinary skill in the art that the disclosed embodiments may also be implemented using multiple requestor-computing devices, multiple database servers, and multiple applications servers, without deviating from the scope of the disclosure.

The separator 202 may be configured to execute a set of instructions stored in the memory unit 110 to perform one or more operations. The separator 202 may be coupled to the memory unit 110, the processor 104, the comparator 204, the transceiver 206, the transcription unit 208, the writing unit 210, the fixed visual capturing unit 212, and the movable visual capturing unit 214.

In an embodiment, a separator 202 may be configured to separate the one or more captured videos in one or more frames of pre-defined lengths.

The comparator 204 may be configured to execute a set of instructions stored in the memory unit 110 to perform one or more operations. The comparator 204 may be coupled to the memory unit 110, the processor 104, the separator 202, the transceiver 206, the transcription unit 208, the writing unit 210, the fixed visual capturing unit 212, and the movable visual capturing unit 214.

In an embodiment, the comparator 204 may be configured to compare each of the separated plurality of frames with the stored plurality of images related to surgical devices and said stored plurality of actions.

The transceiver 206 may be operable to communicate with the one or more devices, such as the comparator 204, the memory unit 110, the processor 104, the separator 202, the transcription unit 208, the writing unit 210, the fixed visual capturing unit 212, the movable visual capturing unit 214 over the communication network 108.

In an embodiment, the transceiver 206 may be operable to transmit or receive the metadata to/from various components of the system environment 100. In an embodiment, the transceiver 206 is coupled to the I/O unit 112 through which the transceiver 206 may receive or transmit metadata/messages/instructions associated with the one or more patients. In an embodiment, the I/O unit 112 may be realized through, but not limited to, an antenna, an Ethernet port, an USB port or any other port that can be configured to receive and transmit data. The transceiver 206 may receive and transmit data/messages in accordance with various communication protocols such as, TCP/IP, UDP, and 2G, 3G, or 4G.

The transcription unit 208 may be configured to execute a set of instructions stored in the memory unit 110 to perform one or more operations. The comparator 204 may be coupled to the memory unit 110, the processor 104, the separator 202, the transceiver 206, the transcription unit 208, the writing unit 210, the fixed visual capturing unit 212, and the movable visual capturing unit 214.

In an embodiment, the transcription unit 208 may be configured to transcript the positive outputs of the comparison of the each of the separated plurality of frames with the stored plurality of images related to surgical devices and said stored plurality of actions.

The writing unit 210 may be configured to execute a set of instructions stored in the memory unit 110 to perform one or more operations. The writing unit 210 may be coupled to the memory unit 110, the processor 104, the separator 202, comparator 204, the transceiver 206, the transcription unit 208, the fixed visual capturing unit 212, and the movable visual capturing unit 214.

In an embodiment, the writing unit 210 may be configured to write the report in a human readable language based on the transcript content.

The fixed visual capturing unit 212 may be configured to execute a set of instructions stored in the memory unit 110 to perform one or more operations. The fixed visual capturing unit 212 may be coupled to the memory unit 110, the processor 104, the separator 202, comparator 204, the transceiver 206, the transcription unit 208, the writing unit 210, and the movable visual capturing unit 214.

In an embodiment, the fixed visual capturing unit 212 may capture one or more videos. The one or more videos may correspond to a video of procedure that may be performed by the medical practitioners on the patient on the operation table. The one or more videos may include sound also.

The movable visual capturing unit 214 may be configured to execute a set of instructions stored in the memory unit 110 to perform one or more operations. The movable visual capturing unit 214 may be coupled to the memory unit 110, the processor 104, the separator 202, comparator 204, the transceiver 206, the transcription unit 208, the writing unit 210, and the fixed visual capturing unit 212.

In an embodiment, movable visual capturing unit 214 may capture one or more videos. The one or more videos may correspond to a video of procedure that may be performed by the medical practitioners on the patient on the operation table. The one or more videos may include sound also.

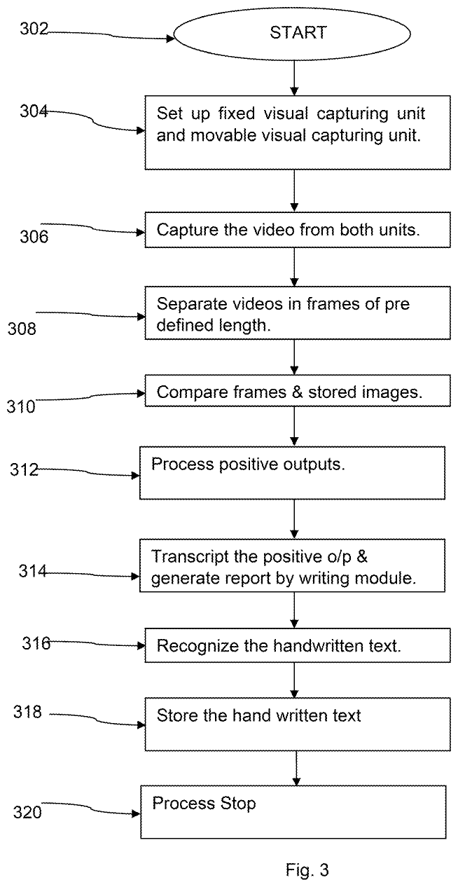

FIG. 3 is a flowchart illustrating a method for surgical image processing and reporting system, in accordance with at least one embodiment. With reference to FIG. 2, there is shown a flow chart 300 and the process starts at step 320 and proceed to step 304.

At step 304, a fixed visual capturing unit 212 and a movable visual capturing unit 214 may be setup, based on type of treatment that the medical practitioners apply on the patient. The patient may correspond to the person who is under treatment by the medical practitioners. Further, the fixed visual capturing unit 212 may correspond to a rectangular prism-shaped visual capturing unit. The prism-shaped visual capturing unit may be placed at a pre-determined location, which may be a location above the operation table where the patient may be treated. Furthermore, the movable visual capturing unit 214 may correspond to an oval shaped capturing unit. The oval-shaped visual capturing unit may be placed at a pre-determined location, which may be a continuous movable location on the head of the medical practitioner.

In an embodiment, the fixed visual capturing unit 212 on the light may be of 3×2×2 in and the movable visual capturing unit 214 on the goggles may be 0.25×0.4×0.25 in. (Length×width×height).

In an embodiment, the fixed visual capturing unit 212 and the movable visual capturing unit 214 may be voice enabled and night vision enabled. The voice enable feature may be utilized to activate the capturing units 212 and 214 to prompt to the direction of the voice. Henceforth, both the placed cameras are real time acting cameras. Furthermore, both the capturing units may have zooming capabilities.

At step 306, one or more videos are captured from the fixed visual capturing unit 212 and the movable visual capturing unit 214. In an embodiment, the fixed visual capturing unit 212 and the movable visual capturing unit 214 may capture one or more videos. The one or more videos may correspond to a video of procedure that may be performed by the medical practitioners on the patient on the operation table. The one or more videos may include sound also.

In an embodiment, the captured one or more videos from the fixed visual capturing unit 212 and the movable visual capturing unit 214 may be processed firstly to remove any distortions and to enhance the clarity of the captured videos.

In an embodiment of the present invention, the fixed visual capturing unit 212 and the movable visual capturing unit 214 may be voice enabled automatically rotatable cameras to capture all incidents happening in the operation room.

At step 308, one or more captured videos are separated in one or more frames of pre-defined lengths. In an embodiment, a separator 202 may be configured to separate the one or more captured videos in one or more frames of pre-defined lengths.

In an embodiment, length of the frames may be pre-defined. This may enable a streamlined communication of the captured videos due to the small bit size. Furthermore, a pre-filter may also be embedded in separating the one or more captured videos. The pre-filters may filter out the videos wherein no action has been performed, which may be a time when the medical practitioners may be changing the dress or attending to some other activity which may not be related to the operation.

At step 310, each of the separated plurality of frames is compared. In an embodiment, each of the separated plurality of frames is compared with the stored plurality of images related to surgical devices and said stored plurality of actions. Furthermore, a comparator 204 may be configured to compare each of the separated plurality of frames with the stored plurality of images related to surgical devices and said stored plurality of actions. In an embodiment, the memory unit 110 may be configured to store a library of images related to surgical devices and a library of plurality of actions performed by the medical practitioners during the operation.

In an embodiment, the memory unit 110 may store the plurality of images related to the surgical devices and the plurality of actions. The plurality of images related to the surgical devices may correspond to the frequently used devices/apparatus by the medical practitioners in the operation theaters. Additionally, the images related to the plurality of actions may correspond to a movement of hand/wrist/face/expressions of the medical practitioners while performing the operation of the patient.

At step 312, based on the comparison of step 310, one or more positive output is processed. In an embodiment, the processor 104 may be configured to process the one or more positive output. In an embodiment, the positive output may correspond to a similar match found between the surgical devices or the action in a frame from each of the separated plurality of frames and the stored plurality of images related to surgical devices or the stored plurality of actions.

At step 314, the positive output is transcript and a report is generated. In an embodiment, the transcription unit 208 may be configured to transcript the positive outputs and thereafter the writing unit 210 may be configured to write the report in a human readable language based on the transcript content.

In an embodiment, an internal tagging of the stored plurality of images related to the surgical devices and the plurality of actions may have been done, so that whenever a positive match is found, then that tag is moved to a temporary generated file in the memory 110 which stores the successfully matched images and frame for further processing by the processor 104.

At step 316, when no match is found at step 310 based on the comparison, then that frame is as recorded is embedded in the report presented at step 314 to the medical practitioner. Now, when the medical practitioner may write about the non matched video, then this may be recorded by the processor 104.

At step 318, the written content by the medical practitioner are saved for later usage and comparisons in the memory unit 110, and the process stops at step 320. In an embodiment of the present invention, the handwritten notes made by the medical practitioners may also be stored in the memory unit 110. This may further be utilized for analyzing the operation performed on the patient.

In an embodiment, the final written report may be displayed. The I/O unit 112 may be configured to display the written report and other relevant data of the patient.

The process ends at step 320.

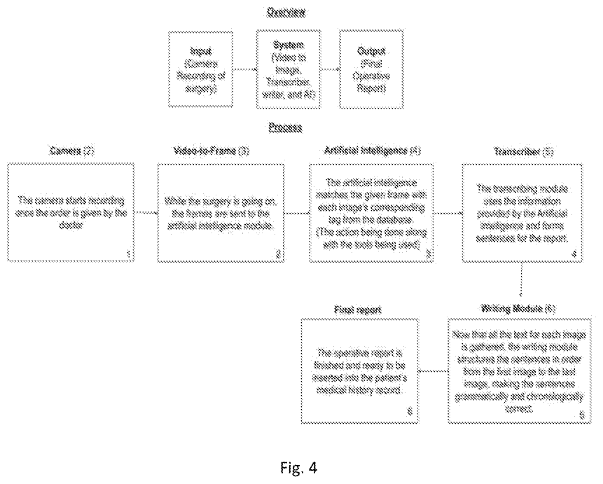

FIG. 4 is an exemplary scenario for surgical image processing and reporting system, in accordance with at least one embodiment.

In an exemplary scenario, the disclosed method is performed by the disclosed system as, a video of the surgery via cameras that are placed at multiple locations is taken. The first camera will be connected to the overhead lights to record surgery from above the surgery table and the camera will be rectangular prism-shaped. The second camera will be oval-shaped and will be connected to surgeon goggles on the side where it will receive an up-close view of the surgery. The camera on the light will be 3×2×2 in. and the camera on the goggles will be 0.25×0.4×0.25 in. (Length×width×height).

The video recorded by the camera is then separated into individual frames, which are analyzed by the artificial intelligence (referred to as AI from here on out) to decode the actions of the doctors and the tools used. The AI is able to do that through a database filled with various images of different possible tools/equipment and the actions that are performed by those tools.

Each stored image in said database has a tag in the form of words in binary. The AI compares the incoming frames from the cameras to the stored images in the database. There will be two separate databases for actions and for tools/equipment. The AI will run through each database with the frame to separately identify the action being done and then the tool through which the action is done. If a match is found in the database, the AI sends the said frame and the corresponding tag to the transcription machine. The tags indicate which tool is being used or which action is being done in the given image so that the transcription module can identify it properly.

Each tag will have thousands of corresponding images from different lightings, angles, and distances. An example would be of a doctor holding a scalpel. This image would have a tag associated with it. If there is no match with any images in the database, the AI will tell the transcription machine that there is no match (this error will be elaborated later on).

The AI sends the tags with the corresponding frames to the Transcription module to be decoded from binary into an English sentence. The tag will have a unique code that the transcription machine can identify as an English sentence. Next, the sentence is sent to the writing module which formats the sentences with necessary periods and other proper punctuation as well as formats it to print in a certain manner.

The final format for the report will be in the following manner: the page will be divided into two columns, the right side of the report will have the transcription from said AI, and the left side of the report contains video clips for the matching transcription so that the Professional can watch and check if the transcription is accurate. The video will also be beneficial if/when the AI receives a frame that is not stored in its database. In such a case, transcription is skipped and only the video will be attached. This unknown part will also have a red underline with it, indicating to the professional reading it that there was something that the AI could not comprehend and attention is needed to fill in the red underline.

The professional can simply watch the associated clip on the left-hand side and fill in what the AI couldn't. This is the true reason that the use of an AI is so essential in the whole process. The AI will see what the doctor has written and learned what was going on the video. It will add it to its database as a new tool/action. This will be a never-ending cycle of the AI continuously learning new images and increasing its default database. Although this invention is specifically designed for surgery and live transcription, it can have a wide variety of uses in almost anything that requires transcription to be done for a procedure taking place.

Various embodiments of the surgical image processing and reporting system have been disclosed. However, it should be apparent to those skilled in the art that modifications in addition to those described, are possible without departing from the inventive concepts herein. The embodiments, therefore, are not restrictive, except in the spirit of the disclosure. Moreover, in interpreting the disclosure, all terms should be understood in the broadest possible manner consistent with the context. In particular, the terms “comprises” and “comprising” should be interpreted as referring to elements, components, or steps, in a non-exclusive manner, indicating that the referenced elements, components, or steps may be present, or utilized, or combined with other elements, components, or steps that are not expressly referenced.

The disclosed methods and systems, as illustrated in the ongoing description or any of its components, may be embodied in the form of a computer system. Typical examples of a computer system include a general-purpose computer, a programmed microprocessor, a micro-controller, a peripheral integrated circuit element, and other devices, or arrangements of devices that are capable of implementing the steps that constitute the method of the disclosure.

The computer system comprises a computer, an input device, a display unit and the Internet. The computer further comprises a microprocessor. The microprocessor is connected to a communication bus. The computer also includes a memory. The memory may be Random Access Memory (RAM) or Read Only Memory (ROM). The computer system further comprises a storage device, which may be a hard-disk drive or a removable storage drive, such as, a floppy-disk drive, optical-disk drive, and the like. The storage device may also be a means for loading computer programs or other instructions into the computer system. The computer system also includes a communication unit. The communication unit allows the computer to connect to other databases and the Internet through an input/output (I/O) interface, allowing the transfer as well as reception of data from other sources. The communication unit may include a modem, an Ethernet card, or other similar devices, which enable the computer system to connect to databases and networks, such as, LAN, MAN, WAN, and the Internet. The computer system facilitates input from a user through input devices accessible to the system through an I/O interface.

In order to process input data, the computer system executes a set of instructions that are stored in one or more storage elements. The storage elements may also hold data or other information, as desired. The storage element may be in the form of an information source or a physical memory element present in the processing machine.

The programmable or computer-readable instructions may include various commands that instruct the processing machine to perform specific tasks, such as steps that constitute the method of the disclosure. The systems and methods described can also be implemented using only software programming or using only hardware or by a varying combination of the two techniques. The disclosure is independent of the programming language and the operating system used in the computers. The instructions for the disclosure can be written in all programming languages including, but not limited to, “C,” “C++,” “Visual C++,” Java, and “Visual Basic.” Further, the software may be in the form of a collection of separate programs, a program module containing a larger program or a portion of a program module, as discussed in the ongoing description. The software may also include modular programming in the form of object-oriented programming. The processing of input data by the processing machine may be in response to user commands, the results of previous processing, or from a request made by another processing machine. The disclosure can also be implemented in various operating systems and platforms including, but not limited to, “Unix,” “DOS,” “Android,” “Symbian,” and “Linux.”

The programmable instructions can be stored and transmitted on a computer-readable medium. The disclosure can also be embodied in a computer program product comprising a computer-readable medium, or with any product capable of implementing the above methods and systems, or the numerous possible variations thereof.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

A person having ordinary skills in the art will appreciate that the system, modules, and sub-modules have been illustrated and explained to serve as examples and should not be considered limiting in any manner. It will be further appreciated that the variants of the above disclosed system elements, or modules and other features and functions, or alternatives thereof, may be combined to create other different systems or applications.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

The claims can encompass embodiments for hardware, software, or a combination thereof.

Although a few implementations have been described in detail above, other modifications are possible. Moreover, other mechanisms for performing the systems and methods described in this document may be used. In addition, the logic flows depicted in the figures may not require the particular order shown, or sequential order, to achieve desirable results. Other steps may be provided, or steps may be eliminated, from the described flows, and other components may be added to, or removed from, the described systems. Accordingly, other implementations are within the scope of the following claims.

What is claimed is:

1. A medical reporting system, said system comprising:
a fixed visual capturing unit integrated at a first location configured to capture one or more first set of visuals;
a movable visual capturing unit integrated at a second location configured to capture one or more second set of visuals;
a memory unit configured to store said captured one or more first set of visuals and said captured one or more second set of visuals;
a separator configured to separate said stored one or more first set of visuals and said stored one or more second set of visuals in plurality of frames of pre-defined length;
a first database unit configured to store plurality of images related to surgical devices;
a second database unit configured to store plurality of actions;
a comparator configured to compare each of said separated plurality of frames, said stored plurality of images related to surgical devices and said stored plurality of actions; and
a processor configured to:
process a positive output of said comparator; wherein said positive output is a similar match found between said surgical devices or said action in a frame from each of said separated plurality of frames and said stored plurality of images related to surgical devices or said stored plurality of actions;
a transceiver configured to communicate said positive output to a transcription unit, wherein said transcription unit is configured to transcript said communicated matched positive output for medical reporting.

2. The medical reporting system as claimed in claim 1 further comprising a display module.

3. The medical reporting system as claimed in claim 1 wherein said first location and said second location are predetermined locations.

4. The medical reporting system as claimed in claim 1 wherein said fixed visual capturing unit is a rectangular prism-shaped unit.

5. The medical reporting system as claimed in claim 1 wherein said movable visual capturing unit is an oval shaped unit.

6. The medical reporting system as claimed in claim 1 wherein said processor is further configured to determine non positive outputs.

7. The medical reporting system as claimed in claim 1 further comprising a content writing unit.

8. A medical reporting method, said method comprising:
capturing, by a fixed visual capturing unit, one or more first set of visuals;
capturing, by a movable visual capturing unit, one or more second set of visuals;
storing, in a memory unit, said captured one or more first set of visuals and said captured one or more second set of visuals;
separating, by a separator, said stored one or more first set of visuals and said stored one or more second set of visuals in plurality of frames of pre-defined length;
storing, in a first database unit, plurality of images related to surgical devices;
storing, in a second database unit, plurality of actions;
comparing, by a comparator, each of said separated plurality of frames, said stored plurality of images related to surgical devices and said stored plurality of actions;
processing, by a processor, a positive output of said comparison; wherein said positive output is a similar match found between said surgical devices or said action in a frame from each of said separated plurality of frames and said stored plurality of images related to surgical devices or said stored plurality of actions;
communicating, by a transceiver, said positive output to a transcription unit; and
transcripting, by a transcription unit, said communicated matched positive output for medical reporting.

9. The medical reporting method as claimed in claim 8, wherein said method further comprising displaying one or more first set of visuals in real time.

10. The medical reporting method as claimed in claim 8, wherein said method further comprising displaying one or more second set of visuals in real time.

11. The medical reporting method as claimed in claim 8 further comprising sorting the compared frames based on said positive outputs.

12. The medical reporting method as claimed in claim 8 further comprises generating a medical report for said medical reporting.

13. The medical reporting method as claimed in claim 8 further comprising generating medical database of a user based on communicated medical reporting.

14. The medical reporting method as claimed in claim 8, further comprising comparing generated medical database of said user with a historical record of said user to record one or more effects of treatments.

15. The medical reporting method as claimed in claim 8 further comprises writing said medical report in one or more user friendly language for medical reporting.
